(19) United States
(12) Patent Application Publication
(10) Pub. No.: US 2022/0159922 A1
(43) Pub. Date: May 26, 2022

(54) PROCESS FOR PRODUCING A GENETICALLY MODIFIED SEED

(71) Applicant: Kah Meng LIM, Singapore (SG)

(72) Inventor: Kah Meng LIM, Singapore (SG)

(21) Appl. No.: 17/602,395

(22) PCT Filed: Apr. 13, 2020
(86) PCT No.: PCT/SG2020/050227

§ 371 Date: Oct. 8, 2021

(30) Foreign Application Priority Data
Apr. 11, 2019 (SG) 10201903274T

(51) Int. Cl. A01H 6/28; C12N 15/82; A01H 4/00; B33Y 80/00

(52) U.S. Cl. CPC A01H 6/28; C12N 15/8213; B33Y 80/00; A01H 4/006; C12N 15/8243

(57) ABSTRACT

This invention relates to a process for producing a genetically modified seed. In particular, there is provided a process for producing a genetically modified Cannabis seed that germinates into a plant, the process comprising: (a) preparing a cell culture comprising genetically engineered Cannabis cells having at least one gene that expresses a psychoactive cannabinoid deleted; (b) establishing a callus culture for forming a somatic embryo; (c) forming a bio-ink comprising the somatic embryo that is encapsulated by a hydrogel and used as an artificial seed; and (d) three-dimensional (3D) printing the artificial seed comprised in the bio-ink. Also provided the 3D-printed artificial Cannabis seed having a shape other than that of a naturally occurring wild type Cannabis seed.

This invention relates to a process for producing a genetically modified seed. In particular, it relates to a process of producing a genetically modified Cannabis seed that germinates into a plant. More particularly, the invention relates to a genetically modified Cannabis seed having a shape other than that of a naturally occurring wild type Cannabis seed.

Cannabis has been used for medicinal purposes throughout history. Cannabis has been shown to provide therapeutic benefits as an appetite stimulant, as an antiemetic, as an analgesic and in the management of various conditions including glaucoma, Parkinson's Disease, Alzheimer's Disease, Multiple Sclerosis and chronic inflammation.

Cannabis contains numerous chemically distinct components many of which have therapeutic properties. The main therapeutic components of medical Cannabis are delta-9-tetrahydrocannabinol (THC) and cannabidiol (CBD).

THC is the primary psychoactive component of Cannabis and has been shown to provide therapeutic benefits as an antiemetic, analgesic and in the management of glaucoma. Conversely, strains of medical Cannabis with high proportions of THC may cause feelings of anxiety and/or disorientation.

CBD is the main non-psychoactive component in Cannabis. CBD is an agonist to serotonin receptors and has been shown to have therapeutic benefit in therapies for neuropathic pain and neural inflammation.

Cannabis (Cannabis sativa) is well known and widely used for the production of medical Cannabis. However, along with key Cannabis compound, tetrahydrocannabinolic acid (THC), Cannabis also produces a range of other secondary metabolites with proven and potential value as pharmaceuticals. However, only low levels are produced within the plant and, thus, high production and purification costs represent the major barriers to commercial viability of these pharmaceuticals. Metabolic engineering of Cannabis secondary metabolite biosynthesis pathways can re-direct biochemical reactions, intermediates and energy from biosynthesis of THC to alternative compounds. This approach can lead to the development of new Cannabis strains with value added production of novel pharmaceuticals.

Various studies and publications show how the cannabinoid molecules can interact with the human endocannabinoid system.

Currently, hemp is legal in some countries but marijuana is illegal in almost all countries. But hemp CBD is low in content to be medically useful (only 3.5%) while marijuana CBD is higher at 20% and would be medically useful.

The fact that some countries permit the widespread farming of industrial hemp is because it has low content of THC less than 0.3% but it is restricted only to industrial applications. CBD has great medical applications. Of late, CBD has been used to make useful medicines, making medical Cannabis or more accurately medical CBD therapies very important and relevant in the future development of effective medicines. One application for treating epilepsy has been obtained USA FDA approval.

There are many hot research all round the world to obtain the best strains of Cannabis via natural selection of breeding but this would be very tedious and slow. Also, there are attempts to use plant stem cells to make plantlets in tissue cultures but little success has been achieved to yield high levels of CBD.

The greatest problem is not about getting enough CBD, the greatest problem is whether we could enable wider global research communities to gain access to these beneficial medicinal crops and to speed up more research breakthroughs on potential cures for chronic diseases and allow these treatments to help the patients all around the world who needed them and not just limited to a few countries or privileged patients who can afford them.

By allowing widespread farming of the beneficial crop in many countries, we could help farmers to turn on an effective global economy, resolving poverty in most countries as non-THC medical marijuana would be a high-income generating crop. And in getting global acceptance, the price of CBD would only go down and this would only be good news to patients around the world as possible medicines from Cannabis would be more affordable due to mass market adoption for production.

The main reason most countries across the whole world regard marijuana as a controlled substance is due to the fact that it contains the psychoactive component, THC and that this would be lead to addiction, drug abuse leading to brain damage to the masses.

Therefore, by removing the harmful component, THC from the marijuana plant, thus our latest invention, we would be able to create marijuana plant with zero THC content but also having highest CBD content possible so as to enable global communities to farm it.

Even though Cannabis has been found to have many medical benefits as more and more research being done by countries who have access to the plant, Cannabis Plant and its products are generally still inaccessible to majority of the world and are deemed illegal by many due to the presence of the psychoactive compound THC. THC can be addictive and overdose of THC could harm and destroy our brain.

The plant has been monopolized by a few countries. As more and more research being carried out to understand the functions and effects of the cannabinoid compounds especially in the area of treatment of chronic diseases eg. chronic pain and incurable neurodegenerative diseases eg. parkinson, dementia, schizophenia, Multiple sclerosis etc.

As more and more countries starts to acknowledge the medical benefits of this plant, they'll slowly move towards legalizing the use of Cannabis for medical treatment. The Cannabis plant has more than 100 cannabinoid compounds, and only a handful is currently being studied. Among those molecules that have been studied, research have found promising medical outcomes for treatments of epilepsy, pain management etc.

There's still a lot of unknown cannabinoid molecules that has not been studied. Cost of medical treatment are exorbitant and the cannabinoid drugs are only accessible to only the wealthy people.

The listing or discussion of an apparently prior-published document in this specification should not necessarily be taken as an acknowledgement that the document is part of the state of the art or is common general knowledge.

Any document referred to herein is hereby incorporated by reference in its entirety.

In a first aspect of the invention, there is provided a process for producing a genetically modified Cannabis seed that germinates into a plant, the process comprising: (a) preparing a cell culture comprising genetically engineered Cannabis cells having at least one gene that expresses a psychoactive cannabinoid deleted; (b) establishing a callus culture for forming a somatic embryo; (c) forming a bio-ink comprising the somatic embryo; and (d) three-dimensional (3D) printing the seed.

In various embodiments, step (a) comprises obtaining cells from a wild-type Cannabis plant and genetically deleting the at least one gene that express psychoactive cannabinoids, wherein the Cannabis plant contains a high level of CBDV content. For example, a suitable wild-type Cannabis plant may be one that has at least 20% amount of non-psychoactive cannabinoid compounds, i.e. CBD, CBDV etc.

In various embodiments, the at least one gene that expresses a psychoactive cannabinoid deleted is a gene that encodes for a psychoactive cannabinoid selected from the group consisting of THCA, THC, THCVA and THCV. Preferably, all genes that express a psychoactive cannabinoid compound is deleted from the cell's genome. Such genes may include any compounds associated with a pathway associated with any one of THCA, THC, THCVA and THCV.

In various embodiments, the gene is the THCA synthase gene.

In various embodiments, the process further comprises the step of replacing the at least one gene that expresses a psychoactive cannabinoid with a reporter gene.

In various embodiments, the at least one reporter gene comprises a detectable label.

In various embodiments, the reporter gene is the firefly luciferase gene.

In various embodiments, the process further comprises encapsulating the somatic embryo.

In various embodiments, the step (d) prints a seed having a shape other than that of a naturally occurring wild type Cannabis seed.

In a second aspect of the invention, there is provided genetically modified Cannabis seed that germinates into a plant, wherein the seed has a shape other than that of a naturally occurring wild type Cannabis seed.

In a third aspect of the present invention, there is provided a plant produced from a seed produced by a process according to the first aspect of the invention, or from a seed according to claim 10.

This invention not only produces a Cannabis cell culture for forming a seed or plant that is free from the harmful effects of or non-legal psychoactive cannabinoids through genetic engineering, but also provides for a process of producing seeds from said genetically engineered Cannabis cell culture through three-dimensional (3D) printing wherein the seeds have shapes other than that of a naturally occurring wild type Cannabis seed. Such shapes include cuboid, triangular, etc.

Advantageously, this invention provides for a process for producing an easily authenticable genetically modified Cannabis plant free from psychoactive cannabinoids content. By producing seeds that have shapes other than that of a naturally occurring wild type Cannabis seed, the invention provides a quick and easy way of identifying and authenticating Cannabis seeds that are safe and legal, i.e. free from psychoactive cannabinoids content. This means that any authentication or identification method for determining whether the seeds are free from psychoactive compounds can be carried out visually at an instance without the need to any lab tests (e.g. genetic) which require more resources such as time and money.

By “Cannabis”, it is meant to refer to all species under this genus and also is used interchangeably here with marijuana and hemp.

By “psychoactive cannabinoids”, it is meant to include compounds such as THCA (Tetrahydrocannabinolic Acid) and THC (tetrahydrocannabinol), THCVA (Tetrahydrocanabivarinic acid), and THCV (Tetrahydrocanabivarinol).

By “non-psychoactive cannabinoids”, it is meant to include compounds such as CBGA (Cannabigerolic acid), CBDA (Cannabidiolic acid), CBCA (Cannabichromenenic acid), CBGVA (Cannabigerovarinic acid), CBDVA (Cannabidivarinic acid), CBCVA (Cannabichromevarinic acid) and CBG (Cannabigerol), CBD (Cannabidiol), CBC (Cannabichromenenol), CBGV (Cannabigerovarinol), CBDV (Cannabidivarinol), and CBCV (Cannabichromevarinol) and others.

There are genetically engineered Cannabis cells that is psychoactive cannabinoid-free or THC-free Cannabis cells; but these lab based methods of production will only deprive the world to have the freedom to grow the agricultural forms by farmers all around the world and also may eradicate the global agricultural economy of Cannabis plants where other plants parts could be produced and be of value to the world. This method of lab production would also eventually result in taking away or promoting the extinction of one important plant from the diminishing diversity of valuable plants in the world.

We hope to enable all people in the world to have greater access to this medically beneficial plant so that more research can be carried out on the plant to uncover more medical breakthrough for the treatment of chronic and neurodegenerative diseases.

By removing the psychoactive cannabinoid component (e.g. THCA, THC, THCVA, THCV) that is deemed to be harmful to the human body, the Cannabis plant would therefore no longer produce THC and THCV, only contain the beneficial non-psychoactive Cannabinoid molecules that can be used for medical treatment and would benefit mankind. Hence, this non psychoactive Cannabis plant would be considered safe for public access.

In order to make this new type of Cannabis plant easily identifiable and traceable, we shall incorporate a biomarker (e.g. a reporter gene with a detectable label) to allow the plant material to be easily detected. The biomarker could be in the form of GFP or luciferase protein expression or any other suitable markers.

This could be in addition to the standard DNA test to determine the genetic sequence of the genetically engineered plant. Furthermore, the Cannabis seeds can be further differentiated using the synthetic seed production method to enhance seed germination as well as distinguishing the appearance of genetically engineered seed material.

Additionally, we could apply 3D bioprinting technology to 3D print the Cannabis seed/cellular material into customisable seed-like shape or structures.

This genetically engineered Cannabis plant would enable farmers from agricultural based countries to farm this genetically engineered Cannabis plant legally and support the global economy especially those from the 3^{rd} world countries by providing job opportunity and income to the unemployed workers, reduce poverty, increase social economy, improve standard of living, improve infrastructures development, reduce abuse and illegal farming of psychoactive marijuana.

In order that the present invention may be fully understood and readily put into practical effect, there shall now be described by way of non-limitative examples only preferred embodiments of the present invention, the description being with reference to the accompanying illustrative figures.

In the Figures:

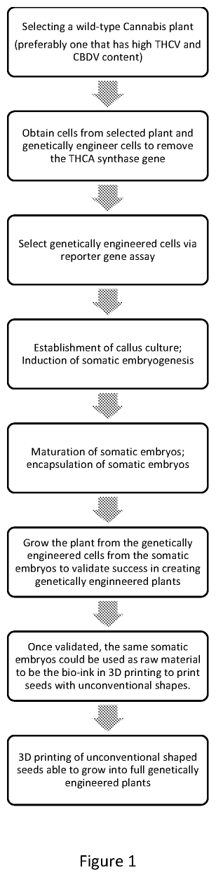

FIG. 1 is a flow chart showing the process of producing a genetically modified Cannabis seed according to an embodiment of the invention;

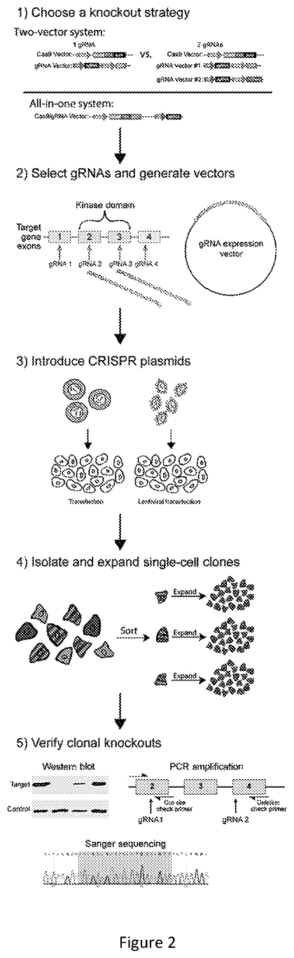

FIG. 2 is a schematic diagram showing the knockout process of the CRISPR method of gene editing;

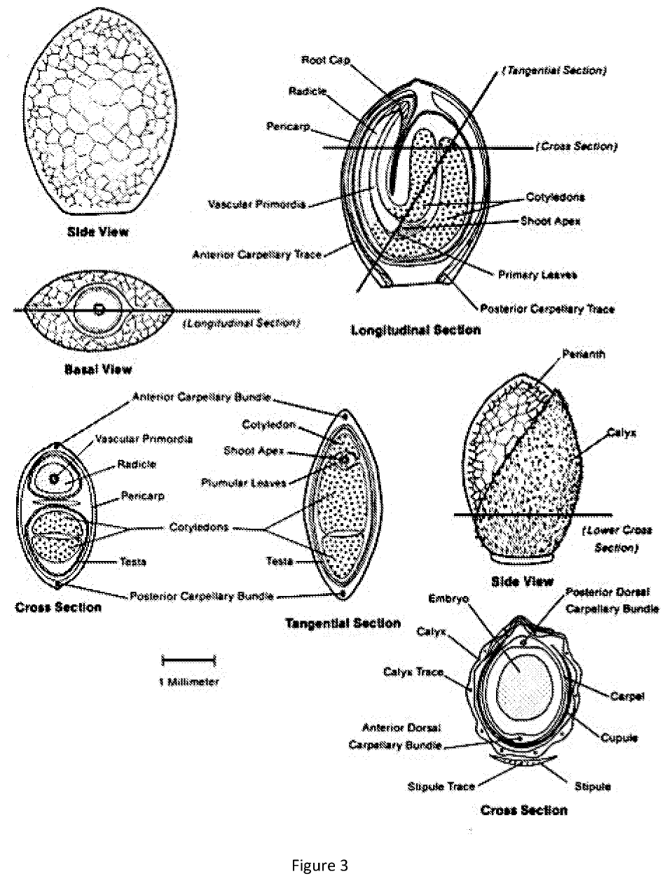

FIG. 3 is a schematic diagram showing a seed's internal layers of cells;

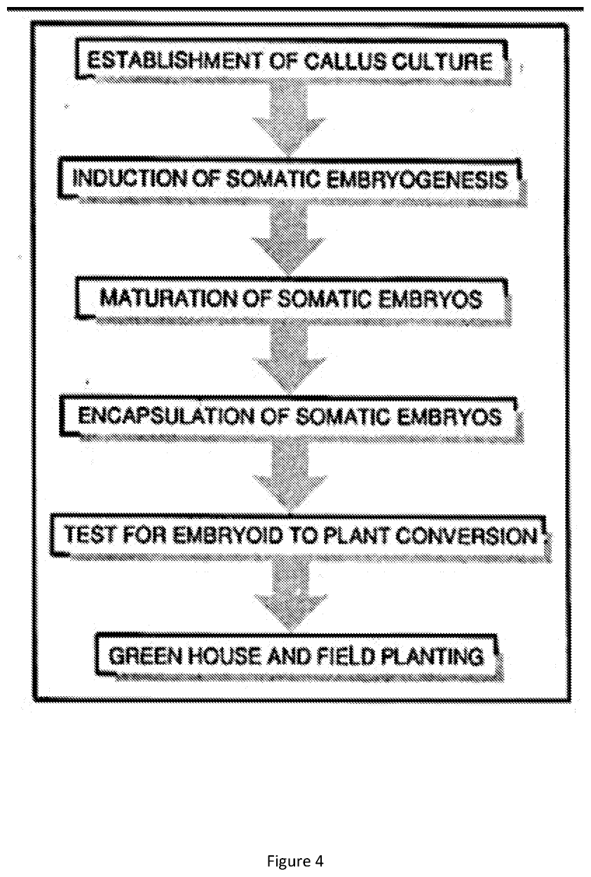

FIG. 4 is a schematic diagram showing the production of an artificial seed (embryoid bodies needed to be the bio-ink materials for the 3D printing of the unique seeds) according to an embodiment of the invention;

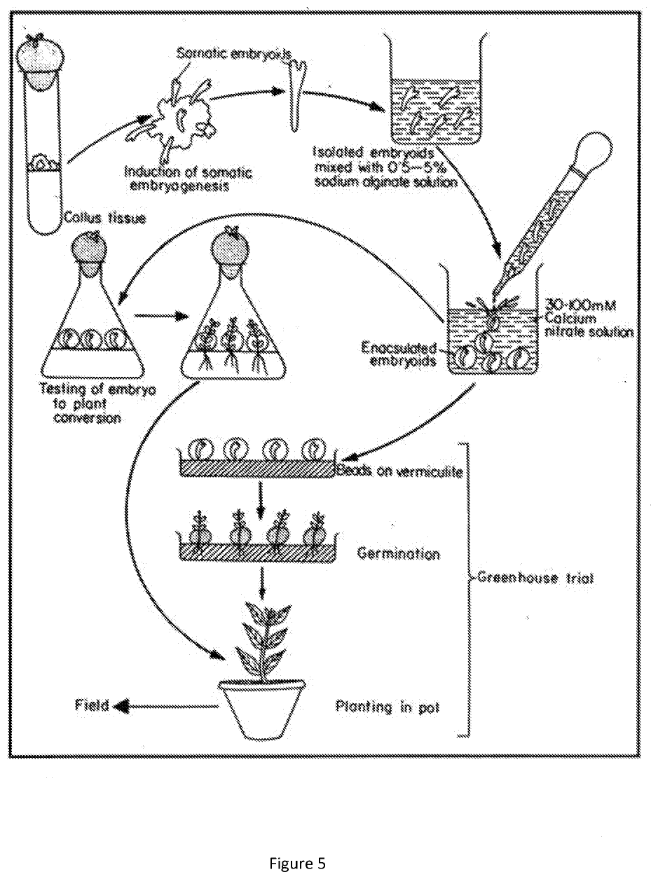

FIG. 5 is a schematic diagram showing the production of an artificial seed (embryoid bodies needed to be the bio-ink materials for the 3D printing of the unique seeds) according to an embodiment of the invention;

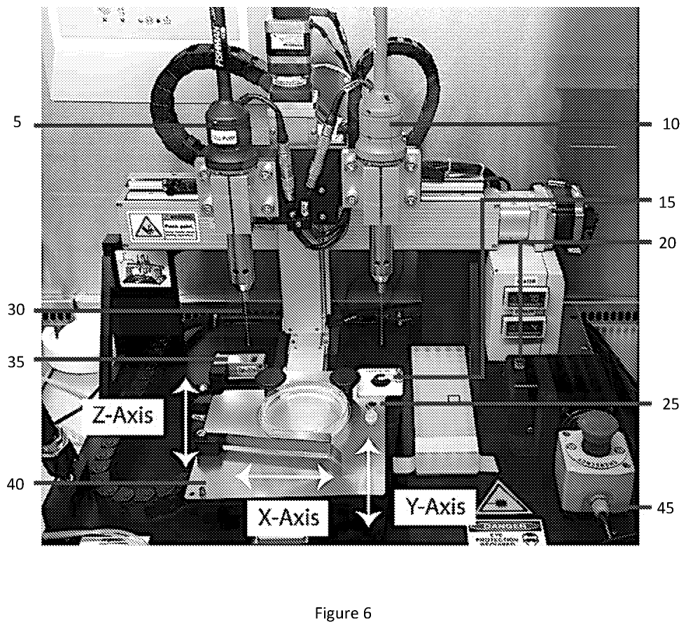

FIG. 6 is a photo of a 3D printer for printing the seed according to an embodiment of the invention;

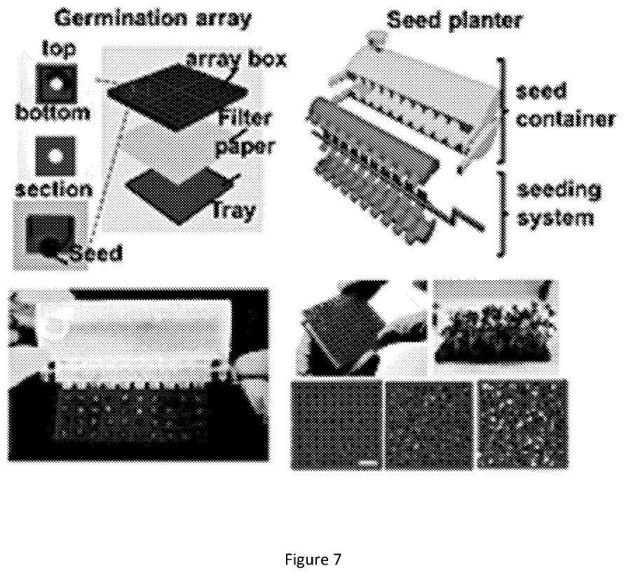

FIG. 7 shows a germination array and seed tray for 3D printing of the seed microenvironment (i.e. the “hardwares”) according to an embodiment of the invention;

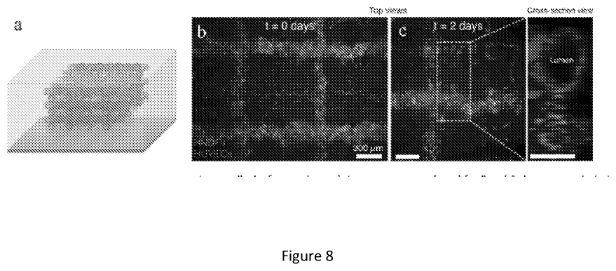

FIG. 8 shows an image of the 3D printing of the 3 layers of the seed microenvironment (the “hardwares”) according to an embodiment of the invention;

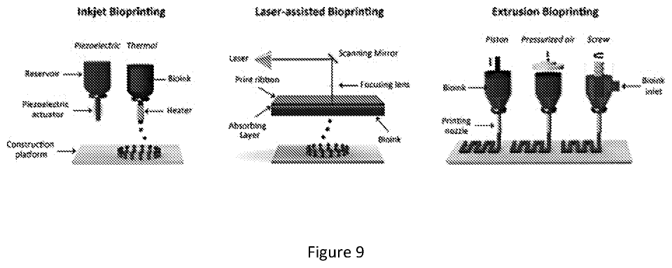

FIG. 9 shows a bioprinting method according to an embodiment of the invention;

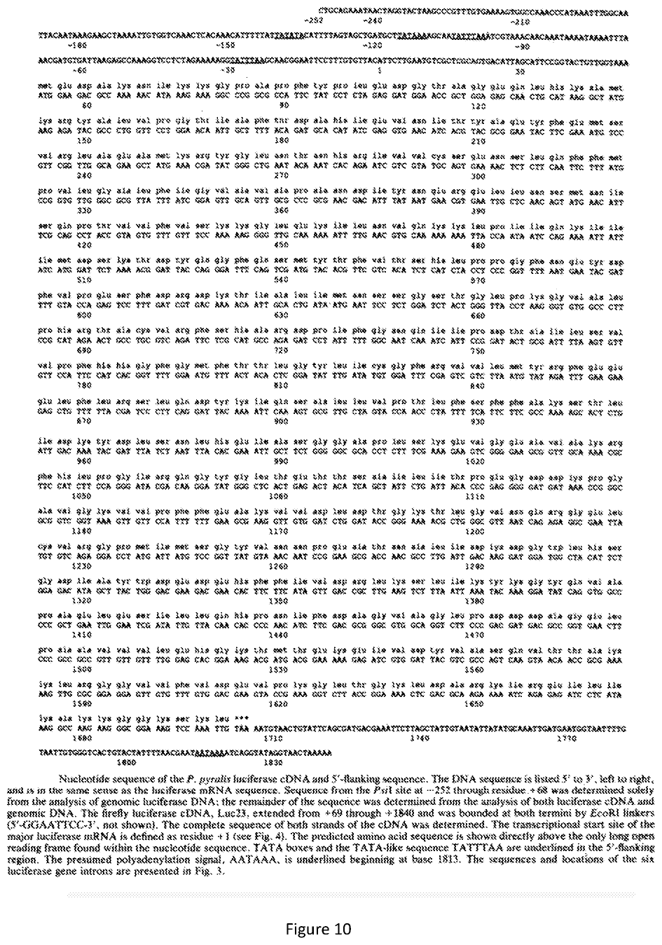

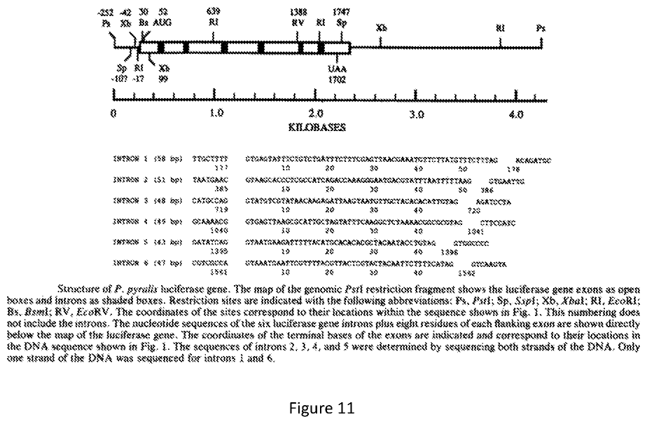

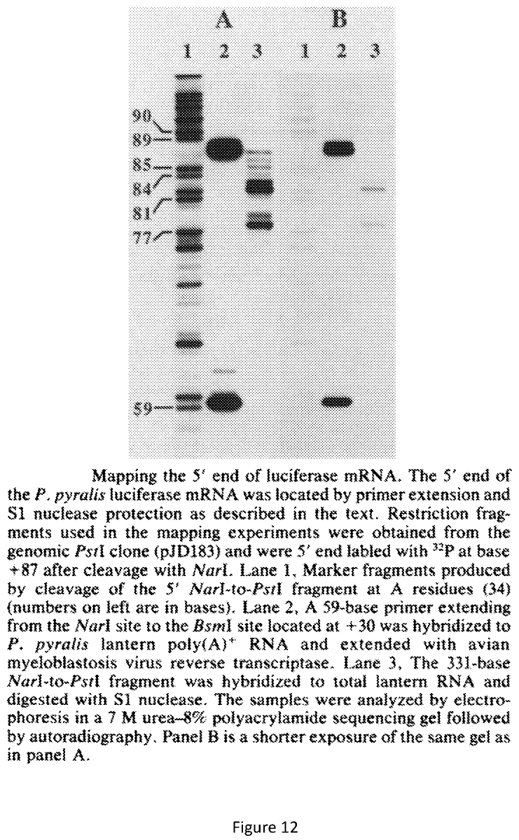

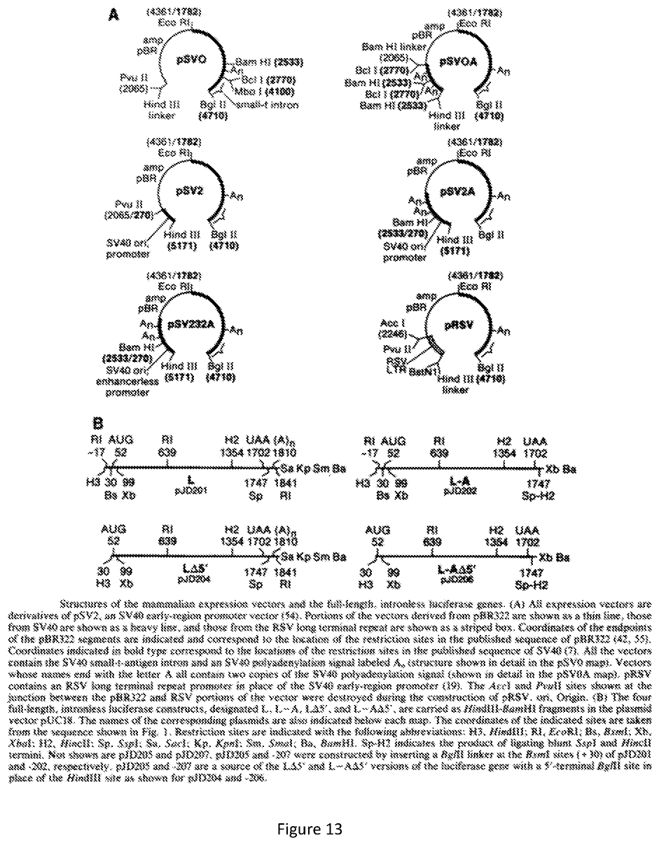

FIGS. 10, 11, 12 and 13 show the various information associated with the firefly luciferase reporter gene;

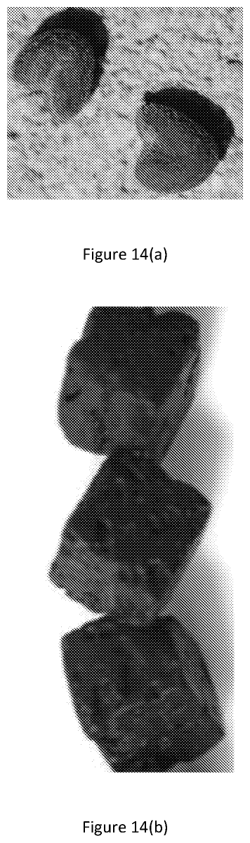

FIGS. 14(a) and (b) are photos showing the printed seed having a heart shape and cuboidal shapes according to an embodiment of the invention; and

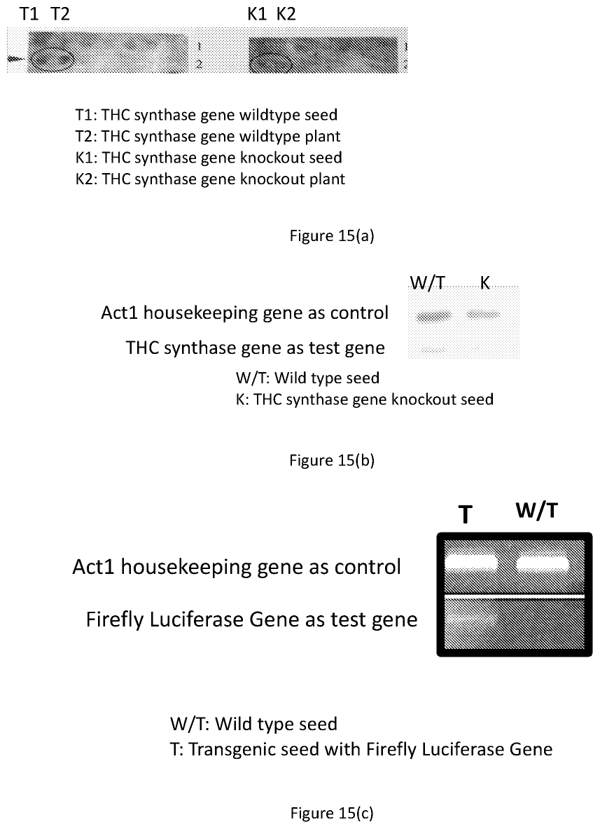

FIGS. 15(a) and (b) show results from Western Blot Assay and PCR carried out to show successful gene deletion of the THC synthase gene, and FIG. 15(c) PCR for a firefly luciferase gene according to an embodiment of the invention.

With reference to FIG. 1, the process of producing a producing a genetically modified Cannabis seed may start with first selecting a wild-type Cannabis plant that exhibits or contents high levels of both psychoactive and non-psychoactive cannabinoid compounds. In various embodiments, a plant that has a high content of both THCV and CBDV is selected. There are various methods known to the skilled person for determining the content of psychoactive and non-psychoactive cannabinoid compounds, for example the use of Western Blot Assay and PCR for our knockout seeds and plants. Please see FIGS. 15(a) and (b) for successful data in the Western Blot Assay and PCR experiments.

Once a suitable plant has been selected, a cell or plant extract is then obtained from said plant so that gene editing using CRISPR gene editing methods are employed to remove or delete those genes that encode for psychoactive cannabinoid compounds. In various embodiments, the plant cells are genetically engineered such as that THCA synthase gene is deleted. For the avoidance of doubt, the invention includes process steps that deletes those genes that encode for THCA, THC, THCVA and/or THCV.

By knowing the specific cDNA sequences of THCA synthase and CBDA synthase (which are only 84% in similarity) within marijuana plant, we would be able to use genetic engineering to remove THCA synthase genes from the genomes of marijuana.

The cells that had the genes that encode for psychoactive cannabinoid compounds successfully deleted are then identified via a reporter gene assay.

These cells are then used to establish a callus culture and somatic embryogenesis is induced. The somatic embryos are matured and then encapsulated, for example with a hydrogel. The encapsulated embryoid bodies is solubilised and then used as the bio-ink for the 3D printing. These genetically engineered artificial seeds are then allowed to grow into a plant and the grown plants are then analysed to validate success.

Once validated, the same somatic embryos could be used as raw material to be the bio-ink that is used in a three-dimensional (3D) printing to print seeds having the genetically engineered genome. In particular, and unique to the invention, is the printing of seeds that have unconventional shapes that is not native to the wild type Cannabis seeds (for example, please see FIGS. 14(a) and (b) showing printed seeds have heart and cuboidal shapes). These 3D printed seeds can then be allowed to grow into full genetically engineered Cannabis plants.

The culture conditions for any cell or tissue growth are standard culture media and conditions known to the skilled person.

As such, the following provides a short summary on the invention.
1. Select marijuana hybrid plant with best cannabinoid profile and growth characteristics
2. Targeted deletion of THCA synthase gene and insertion of reporter gene(s) into the same locus.
3. To drive the cannabinoid synthesis pathway towards the divarinic acid pathway instead of the olivetolic acid pathway
4. Targeted inactivation of hexanoyl-CoA synthetase or olivetolic acid cyclase
5. Targeted insertion of aldehyde dehydrogenase or Enoyl-CoA hydratase
6. The plant cells that express the reporter gene will be the one without THCA gene ie. THC and THCV free marijuana plant.
7. Encapsulation of marijuana plant cells/seeds using synthetic seed production method.
8. Using 3D printing technology to generate a uniform customisable seed structures based on the GM THC Free (non-psychoactive) Marijuana plant cellular materials to create a uniquely identifiable GM THC Free Marijuana seeds products.

### EXAMPLE

The disclosure of PCT application number PCT/IB2016/000814 is incorporated herein by reference.

Described are genetically modified Cannabis plants and Cannabis plant derived products as well as expression cassettes, vectors, compositions, and materials and methods for producing the same. In particular, the present invention relates to a method of making a genetically modified marijuana plants that is free from THC and THCV and is easily authenticable by deleting and replacing the THCA synthase gene with a reporter gene cassette.

Described are certain embodiments of enhancing production of one or more secondary metabolites by downregulation of the production of one or more metabolites having a shared biosynthetic pathway. Certain embodiments provide methods of enhancing production of one or more secondary metabolites that share steps and intermediates in the THC biosynthetic pathway by removing of THC production. In specific embodiments, there are provided methods of enhancing production of CBD and/or Cannabichromene by removing the production of THC. The diagram below shows the biosynthetic pathway.

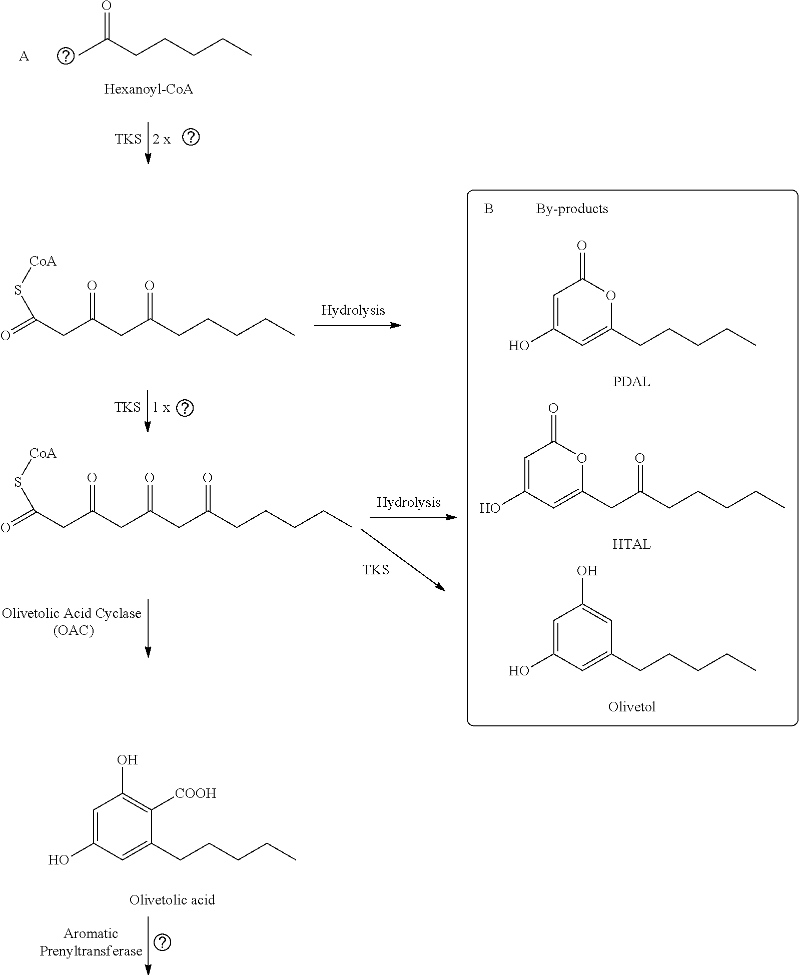

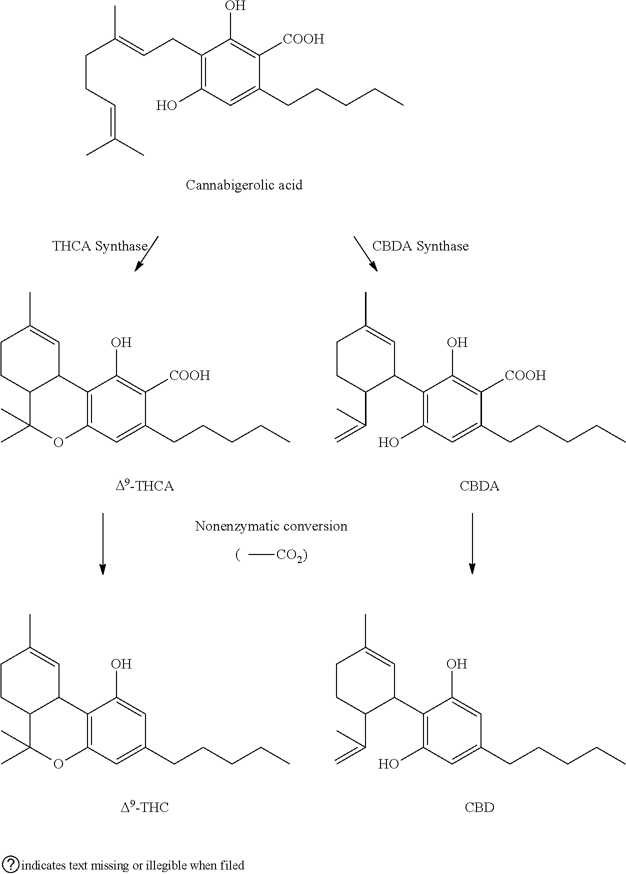

Disruption in the production of THC, CBD, or Cannabichromene will enhance production of the remaining metabolites in this shared pathway. For example, production of CBD and/or Cannabichromene is enhanced by removing production of THC. THC production will be removed by removing the expression and/or activity of tetra hydrocannabinolic acid (THCA) synthase enzyme. Similarly, it will also disrupt the production of THCV and enhance production of the other metabolites in the shared pathway.

Also provided are plants and plant cells having modified production of one or more metabolites having a shared biosynthetic pathway. In certain embodiments, there are provided Cannabis plants and cells enhanced production of one or more secondary metabolites and downregulation of one or more other metabolites having a shared biosynthetic pathway. In certain embodiments, there are provided Cannabis plants and cells having enhanced production of one or more secondary metabolites and downregulation of one or more other metabolites in the THC and THCV biosynthetic pathway.

In certain embodiments, there are provided Cannabis plants and cells having enhanced production of one or more secondary metabolites in the THC and THCV biosynthetic pathway and no THC production.

In specific embodiments, there are provided Cannabis plants and cells having enhanced production of CBD and/or Cannabichromene and no THC production.

Certain embodiments provide for Cannabis plants and/or cells having enhanced production of one or more secondary metabolites that share steps and intermediates in the THC and THCV biosynthetic pathway and no expression and/or activity of THCA synthase. In specific embodiments, there are provided Cannabis plants and/or cells having enhanced production of CBD and/or Cannabichromene and downregulated expression and/or activity of THCA synthase.

### Definitions

In the description and tables herein, a number of terms are used. In order to provide a clear and consistent understanding of the specification and claims, the following definitions are provided. Unless otherwise noted, terms are to be understood according to conventional usage by those of ordinary skill in the relevant art. Where a term is provided in the singular, the inventors also contemplate aspects of the invention described by the plural of that term.

As used herein, the term “expression cassette” refers to a DNA molecule that comprises a selected DNA to be transcribed. In addition, the expression cassette comprises at least all DNA elements required for expression. After successful transformation, the expression cassette directs the cell's machinery to transcribe the selected DNA to RNA. In certain embodiments, the expression cassette expresses an dual sgRNA, that stop the expression of a THCA synthase by deleting its gene.

Different expression cassettes can be transformed into different organisms including bacteria, yeast, plants, and mammalian cells as long as the correct regulatory sequences are used.

As used herein, the term “expression” refers to the combination of intracellular processes, including transcription and translation undergone by a coding DNA molecule such as a structural gene to produce a polypeptide.

As used herein, the term “genetic transformation” refers to process of introducing a DNA sequence or construct (e.g., a vector or expression cassette) into a cell or protoplast in which that exogenous DNA is incorporated into a chromosome or is capable of autonomous replication.

As used herein, the term “heterologous” refers to a sequence which is not normally present in a given host genome in the genetic context in which the sequence is currently found. In this respect, the sequence may be native to the host genome, but be rearranged with respect to other genetic sequences within the host sequence. For example, a regulatory sequence may be heterologous in that it is linked to a different coding sequence relative to the native regulatory sequence.

As used herein, the term “transgene” refers to a segment of DNA which has been incorporated into a host genome or is capable of autonomous replication in a host cell and is capable of causing the expression of one or more coding sequences.

Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was transformed with the DNA segment.

As used herein, the term “transgenic plant” refers to a plant or progeny plant of any subsequent generation derived therefrom, wherein the DNA of the plant or progeny thereof contains an introduced exogenous DNA segment not naturally present in a non-transgenic plant of the same strain. The transgenic plant may additionally contain sequences which are native to the plant being transformed, but wherein the “exogenous” gene has been altered in order to alter the level or pattern of expression of the gene, for example, by use of one or more heterologous regulatory or other elements.

As used herein, a first nucleic-acid sequence, selected DNA, or polynucleotide is “operably” connected or “linked” with a second nucleic acid sequence when the first nucleic acid sequence is placed in a functional relationship with the second nucleic acid sequence. For instance, a promoter is operably linked to an RNA and/or protein-coding sequence, if the promoter provides for transcription or expression of the RNA or coding sequence. Generally, operably linked DNA sequences are contiguous and, where necessary to join two protein-coding regions, are in the same reading frame.

As used herein, the term “transcript” corresponds to any RNA that is produced from a gene by the process of transcription. A transcript of a gene can thus comprise a primary transcription product which can contain introns or can comprise a mature RNA that lacks introns.

As used herein, “nucleases” means natural and engineered (i.e. modified) polypeptides with nuclease activity such as endonucleases possessing sequence motifs and catalytic activities of the “LAGLIDADG,” “GIY-YIG,” “His-Cys box,” and HNH families (e.g. Chevalier and Stoddard, 2001), as well as zinc finger nucleases (ZFNs), naturally occurring or engineered for a given target specificity (e.g. Durai et al., 2005; U.S. Pat. No. 7,220,719), among others. Another contemplated endonuclease is the Saccharomyces cerevisiae HO nuclease (e.g. Nickoloff et al, 1986), or variant thereof.

As used herein, a “custom endonuclease” means an endonuclease that has been evolved or rationally designed (e.g. WO06097853, WO06097784, WO04067736, or US20070117128) to cut within or adjacent to one or more recognition sequences. Such a custom endonuclease would have properties making it amenable to genetic modification such that its recognition, binding and/or nuclease activity could be manipulated.

As used herein, an “allele” refers to an alternative sequence at a particular locus; the length of an allele can be as small as 1 nucleotide base, but is typically larger. Allelic sequence can be denoted as nucleic acid sequence or as amino acid sequence that is encoded by the nucleic acid sequence. Alternatively, an allele can be one form of a gene, and may exhibit simple dominant or recessive behavior, or more complex genetic relationships such as incomplete dominance, co-dominance, conditional dominance, epistasis, or one or more combinations thereof with respect to one or more other allele(s).

A “locus” is a position on a genomic sequence that is usually found by a point of reference; e.g., a short DNA sequence that is a gene, or part of a gene or intergenic region. The loci of this invention comprise one or more polymorphisms in a population; i.e., alternative alleles present in some individuals.

Selecting the Hybrid Plant

Selecting a hybrid strain of marijuana plant that is equally high in both THC and CBD contents Eg. Cannatonic strain. Examples and the listings of the strains can be found here: https://www.medicalmarijuanainc.com/top-5-high-cbd-high-thc-cannabis-strains/https://www.marijuanabreak.com/5-best-11-thc-to-cbd-marijuana-strains

Alternatively, we could also select for hybrid strains that is equally high in THCV and CBDV (https://www.civilized.life/articles/cannabis-strains-high-levels-of-tetrahydrocannabivarin/). A hybrid strain of Marijuana plant (Cannabis Sativa×Indica) has been shown to produce highest THC and CBD content on average (https://www.leafly.com/news/cannabis-101/indica-vs-sativa-which-produces-more-cbd-thc). It would also be beneficial to select for Cannabis hybrid strain with Ruderalis quality due to its cultivation (https://www.rovalqueenseeds.com/blog-top-fastest-growing-cannabis-seeds-by-categories-n519). Advantages include faster growth, auto-flowering etc.

Gene Editing/Deletion

The contents of the paper “Giuliano, C. J. Lin, A., Girish, V., & Sheltzer, J. M. (2019). Generating single cell-derived knockout clones in mammalian cells with CRISPR/Cas9. Current Protocols in Molecular Biology, 128, e100. Doi: 10.1002/cpmb.100” is incorporated herein by reference. It sets out the CRISPR protocol.

The CRISPR system initially evolved as a nucleic acid-targeting bacterial defense mechanism capable of conferring resistance to viral infection (Barrangou et al., 2007). It has since been co-opted by scientists as a means to generate sequence-specific double-strand breaks (DSBs) and to induce other precise alterations in the genomes of cells and organisms (Cong et al., 2013). CRISPR has been particularly useful in the study of mammalian genetics and cell biology, as mammalian somatic cells have historically proven to be highly refractory to genetic modification (Komor, Badran, & Liu, 2017). By expressing the Cas9 nuclease and a suitable guide RNA (gRNA) in mammalian cells, a double-strand break can be introduced at a locus of interest. The cell then has multiple options for repairing that break. If a suitable template is provided, the cell can use homology-directed repair to integrate a novel allele or transgene at the targeted site (Ceasar, Rajan, Prykhozhij, Berman, & Ignacimuthu, 2016). Alternately, the cell can repair the lesion via nonhomologous end joining (NHEJ), an error-prone process that commonly results in an insertion or deletion (indel) mutation at the DSB location (Brinkman et al., 2018). In this way, CRISPR can be used to introduce stable, nonrevertible alterations to mammalian genes. Below, we describe an efficient method to use CRISPR to generate knockout clones in mammalian somatic cell lines.

The protocol is divided into five sections, as outlined below:

1. Choosing a knockout strategy;

FIG. 2 shows an outline of the knockout strategy.

2. Selecting gRNA target sites and performing vector cloning (all target genes listed in the initial submitted document could have their sequences obtained from the weblinks given below);

3. Introducing gRNAs by transfection or transduction;

4. Isolation and expansion of single-cell clones;

5. Knockout verification by western blot analysis, PCR, and/or Sanger sequencing.

Using the dual sgRNA/Cas9 CRISPR gene editing method as reported by Xie et al., 2016. An alternative strategy for targeted gene replacement in plants using a dual-sgRNA/Cas9 design. Nature's Scientific Reports volume 6, Article number: 23890 https://www.nature.com/articles/srep23890 or other similar methods known to the PSA.

To design the dual-sgRNAs CRISPR/Cas9 constructs having dual-sgRNA sequences flanking both 5′ and 3′ ends of the THCA synthase gene:
(a) Design a donor vector carrying a reporter gene eg. eGFP or luciferase gene target to completely replace the THCA synthase genes.
(b) Using the CRISPR/cas9 technology, both the 5′ and 3′ end of the THCAS gene will be cut by the cas9 nucleases causing a DSB. The reporter gene expression cassette is then inserted into the targeted locus in place of the THCAS gene through homology-directed repair activities.

Hence, the successfully edited Cannabis plant will no longer express the THCAS gene, and therefore, will no longer produce the psychoactive compounds in the plant. Furthermore, the engineered marijuana plant can be easily detected and authenticated using the Reporter genes eg. GFP under fluorescent light or luciferase using luminol.

The following is an illustration of the design of an alternative strategy for targeting gene replacement at the AtTFL1 locus using a dual-sgRNA/Cas9 design.

Similar dual-sgRNA/Cas9 gene deletion method can also be applied to any other cannabinoid synthases as well in order to increase the yield of the other non-psychoactive cannabinoid compounds.

In addition to the above, we also provide a method to genetically engineered the marijuana to stop the production of cannabinoids from the olivetolic acid pathways, instead direct the production of cannabinoids to the divaricinc acid pathway. This will allow the increased in production of divarinic derived cannabinoids, for example CDBV, CBCV, CBGV so that more of such compounds can be made available for further research work to understand their medical benefits.

In order to disrupt the olivetolic acid pathway, we will target the hexanol-CoA synthetase enzyme (CsAAE1 gene) involved in the upstream conversion of hexanol to hexanol CoA as Reported in Stout, Joke M. et al. “The hexanoyl-CoA precursor for cannabinoid biosynthesis is formed by an acyl-activating enzyme in Cannabis sativa trichomes.” The Plant journal: for cell and molecular biology 71 3 (2012): 353-65.

Using the CRISPR/Cas9 technology or the dual-sgRNA/Cas9 method discussed earlier, we will be able to disrupt or delete the CsAAE1 gene thus disrupting the olivetolic acid pathway, hence the olivetolic derived cannabinoids eg. CBD, CBC, CBG. (https://www.semanticscholar.org/aper/The-hexanoyl-CoA-precursor-for-cannabinoid-is-by-an-Stout-Boubakir/fafdc68adbf8bfb132eb700cfc9d44d47d866f30)

Alternatively, we could also target the olivetolic acid cyclase gene to prevent conversion of Hexanoyl-CoA into olivetolic acid. (https://www.brenda-enzymes.org/enzyme.php?ecno=4.4.1.26 #UNIPROT)

Alternatively or in additionally, to disrupt the olivetolic pathway, we could also insert and express the AdhE2, aldehyde dehydrogenase gene to convert hexanoyl CoA into 1-hexanol. (https://www.ncbi.nlm.nih.gov/pubmed/21707101)

Or, we could also insert and express Enoyl-CoA hydratase gene to convert hexanoyl CoA to acetyl coA. Once the insertion is successful, we will be able to disrupt the olivetolic pathway. We could include any other enzymes that could breakdown or convert hexanoyl CoA into other derivatives.

The DNA and Peptide Sequences of interest to the invention can be found here:

THCA Synthase

https://www.uniprot.org/uniprot/Q8GTB6

https://www.ncbi.nlm.nih.gov/labs/pubmed/15190053-the-gene-controlling-marijuana-psychoactivity-molecular-cloning-and-heterologous-expression-of-delta1-tetrahydrocannabinolic-acid-synthase-from-Cannabis-sativa-I/?i=2&from=/16143478/related

Hexanoyl CoA Synthetase

https://www.uniprot.org/uniprot/H9A1V3

Olivetolic Acid Cyclase

https://www.brenda-enzymes.org/sequences.php?ID=180962

Aldehyde Dehydrogenase

https://www.uniprot.org/uniprot/Q9ANR5

Enoyl-CoA Hydratase

https://www.uniprot.org/uniprot/?query=Enovl-CoA+hvdratase+&sort=score

The deleted gene may be replaced with a reporter gene, which may be a “2-in-1” reporter gene with detectable label. In various embodiments, the reporter gene is the firefly luciferase gene.

The nucleotide sequence of the luciferase gene from the firefly Photinus pyralis was determined from the analysis of cDNA and genomic clones. The gene contains six introns, all less than 60 bases in length. The 5′ end of the luciferase mRNA was determined by both Si nuclease analysis and primer extension. Although the luciferase cDNA clone lacked the six N-terminal codons of the open reading frame, we were able to reconstruct the equivalent of a full-length cDNA using the genomic clone as a source of the missing 5′ sequence. The full-length, intronless luciferase gene was inserted into mammalian expression vectors and introduced into monkey (CV-1) cells in which enzymatically active firefly luciferase was transiently expressed. In addition, cell Unes stably expressing firefly luciferase were isolated. Deleting a portion of the 5′-untranslated region of the luciferase gene removed an upstream initiation (AUG) codon and resulted in a twofold increase in the level of luciferase expression. The ability of the full-length luciferase gene to activate cryptic or enhancerless promoters was also greatly reduced or eliminated by this 5′ deletion. Assaying the expression of luciferase provides a rapid and inexpensive method for monitoring promoter activity. Depending on the instrumentation employed to detect luciferase activity, we estimate this assay to be from 30- to 1,000-fold more sensitive than assaying chloramphenicol acetyltransferase expression.

FIGS. 10 to 13, and Table 1 provide further details on the luciferase reporter gene.

TABLE 1
Relative levels of transient expression of the
luciferase and CAT genes in CV-1 cells^{a}

| | | Gene expressed | | | | |
|---|---|---|---|---|---|---|
| | Vector | L | L-A | LΔ5′ | L-AΔ5′' | CAT |
| | pSV2 | 100 | 33.6 | | | 100 |
| | pSV0 | 14.2 | 8.8 | 3.8 | 3 | <0.5^{b} |
| | pSV0A | 0 | 0 | 0 | 0 | <0.5 |
| | pSV2A | 73 | | 156 | 134 | 129 |
| | pSV232A | 8.2 | | 1 | 2.6 | 2.9 |
| [symbol] | pRSV | 250 | | | | 300^{c} |

^{a}Levels of luciferase expression were normalized relative to pSV2/L, defined as 100%. Levels of CAT expression were normalized relative to pSV2CAT, defined as 100%. Each value is the average of the results of at least four independent transfection experiments. In parallel transfections of duplicate plates of cells, the absolute number of light units produced by a given luciferase expression vector varied by less than ±15%. [symbol]
indicates data missing or illegible when filed

Making the Seed

FIG. 3 shows a seed microenvironment which the invention sets out to achieve.

The seed microenvironment is the surrounding of the seed that is needed for proper germination, including the scaffolds of supplying nutrients and precursor cells other than the plant stem cells (embroid cells). The seed would also need a good soil composition as part of the seed microenvironment, as follows:

Soil Composition
Water retention: 50% to 70% moisture
pH value of 5.8-6.3
Nutrients: organic substances such as humus, compost, worm castings, guano, etc.

Microorganisms in the soil: mycorrhizal fungi (20%), actinomycetes (30%), diazotrophic bacteria (50%)

We would create a stem cell-other precursor cell coculture system to study intercellular interactions in a model that is more representative of the endogenous 3D microenvironment than conventional 2D cultures. The method can reliably seed primary cells within a bioprinted scaffold fabricated from our scaffolding Bioink.

Artificial seeds are the living seed-like structure which are made experimentally by a technique where somatic embryoids derived from plant tissue culture are encapsulated by a hydrogel and such encapsulated embryoids behave like true seeds if grown in soil and can be used as a substitute of natural seeds.

The following steps are involved in the production of an artificial seed.

(1) Establishment of callus culture

(2) Induction of somatic embryogenesis in callus culture

(3) Maturation of somatic embryos

(4) Encapsulation of somatic embryos

After encapsulation, the artificial seeds are tested by two steps:

(1) Test for embryoid to plant conversion

(2) Green-house and field planting.

Maturation of somatic embryos means the completion of embryo development through some stages. Initially, embryo develops as globular-shaped stage, then heart-shaped stage and finally torpedo-shaped stage. In the final stage, embryo attains maturity and develops the opposite poles for shoot and root development at the two extremities.

This embryo then starts to germinate and produces plantlet. However, in some plant species, such sequential development may not be followed. Again, in some species requiring cold treatment for embryo germination, it may be necessary to chill young or mature embryos for their normal maturation and development into plantlets.

Application of GA3 is also required for root and shoot development during embryo germination in citrus. Water soluble hydrogels have been found suitable for making artificial seeds. A list of some useful hydrogels for encapsulation of somatic embryos are given in Table 8.1.

TABLE 8.1
Useful Hydrogel for Encapsulation

| | | Conc. | | Cone. |
|---|---|---|---|---|
| Gel | | % W/V | Complexing agents | mM |
| 1. | Sethum Alginate | 0.5-5.0 | Calcium salts | 30-100 |
| 2. | Sodium Alginate | 2.0 | Calcium Chloride | 30-100 |
| | with Gelatin | 5.0 | | |
| 3. | Carragenan with | 8 | Potasium or | 500 |
| | Locust Beam Gum | 0.4-1.0 | Ammonium chloride | |
| 4. | Gel-rite ™ | 0.25 | Temperature lowered | |

Two standardized methods have been used to coat somatic embryos:

(i) Gel complexation via a dropping procedure;

(ii) Molding.

In the first method, isolated somatic embryos are mixed with 0.5 to 5% (W/V) Sodium alginate and dropped into 30-100 μM Calcium nitrate solution. Surface complexation begins immediately and the drops are gelled completely within 30 minutes (see FIG. 5).

In the second method, isolated somatic embryos are mixed in a temperature-dependent gel such as Gel-rite and placed in the well of a micro-titer plate and it forms gel when the temperature is cooled down.

To achieve the satisfactory results, research is required in several areas for making artificial seeds. Somatic embryos need to be produced on a large scale, matured to a stage where germination will be at a high rate and frequency and encapsulated embryos will probably need to be coated to prevent capsule desiccation and allow for singulation during planting.

After encapsulation, initially, the effect of coating on somatic embryos is very difficult to assess because the germination and continued development of the encapsulated embryos are sometimes very inconsistent after planting into soil.

So, to overcome this problem, embryo response in terms of embryo to plant development or conversion is tested under aseptic conditions. Embryo conversion frequency is the percent of the somatic embryos that produce green-plants having a normal phenotype.

Embryo to plant conversion includes the following steps:

(i) Encapsulated embryos are placed aseptically on simply agar medium with minimal nutrients.

(ii) Uniform germination of somatic embryos and growth and development of root and shoot systems.

(iii) Production of true leaves.

(iv) Absence of hypstrophy of the hypocotyl.

(v) A green-plant with a normal phenotype.

This assay should be very critical before showing the artificial seed in green-house or in the field. Otherwise, some modifications are to be required. The final assessment will be the green-house or field performance of artificial seed and their yield in comparison to plants derived from true seeds.

Storage of artificial seeds is a great limitation. When the artificial seeds are stored at low temperature, the embryos show a characteristic drop in conversion. The limited storage time of artificial seeds is probably due to an anaerobic environment in the capsule.

This is a problem for somatic embryos because they are not develop-mentally arrested and continue very active respiration. To overcome this limitation, two possible solutions are, to have a smaller ratio of capsule volume to embryo volume so that gas diffusion can readily occur or to induce an arrested state in the embryo using growth control agent in the encapsulation medium.

Although the initial cost for artificial seeds i.e. cost of labour and material for the tissue culture processes and encapsulation, is considerably higher than that for true seeds, still there may be some advantages for the use of artificial seeds.

This embryoid material would include: Validated and tested selected clone embryo materials (see given below diagram as ‘encapsulated embryoid bodies’) proven to be able to convert and grow into plants plus 0.5-5% sodium alginate solution plus 30-100 mM calcium nitrate solution. This is a‘software’ because it contains all the necessary information and instructions for a ‘unique seed’ to be able to germinate into the selected genetically engineered plant which we have earlier designed to be.

Once the ‘Embryo to plant conversion’ has been validated to be successful, that embryo contents or compositions would be used as the composition of the Bio Ink (including the reporter gene to indicate a successful genetic recombinant has been made with the desired genes deleted) to be used for subsequent 3D printing.

3D Printing the Seed

The disclosure contained in US patent publication number 20180184702, and “3D bioprinting of vascularized, heterogeneous cell-laden tissue constructs” Kolesky et al Advanced Materials 2014, Materials Science, Medicine DOI:10.1002/adma.201305506, are incorporated herein by reference.

We developed an appropriate 3D printer (as shown in FIG. 6) that prints a plant mineral nutrients material and seed mixture into customisable shapes. If you gently water the printed seeds, the seeds will germinate.

A new bioprinting method is reported for fabricating 3D tissue constructs replete with vasculature, multiple types of cells, and extracellular matrix. These intricate, heterogeneous structures are created by precisely co-printing multiple materials, known as bioinks, in three dimensions. These 3D micro-engineered environments open new-avenues for drug screening and fundamental studies of wound healing, angiogenesis, and stem-cell niches.

Three-dimensional (3D) in vitro modeling is increasingly relevant as two-dimensional (2D) cultures have been recognized with limits to recapitulate the complex endogenous conditions in the plant body. Additionally, fabrication technology is more accessible than ever. Bioprinting, in particular, is an additive manufacturing technique that expands the capabilities of in vitro studies by precisely depositing cells embedded within a 3D biomaterial scaffold that acts as temporary extracellular matrix (ECM). More importantly, bioprinting has vast potential for customization. This allows users to manipulate parameters such as scaffold design, biomaterial selection, and cell types, to create specialized biomimetic 3D systems. The development of a 3D system is important to recapitulate the seed microenvironment. Plant stem cells, a key population within the seed, are known to communicate with other precursor cells to aid in their transition into germination.

We would create a stem cell-other precursor cell coculture system to study intercellular interactions in a model that is more representative of the endogenous 3D microenvironment than conventional 2D cultures. The method can reliably seed primary cells within a bioprinted scaffold fabricated from CELLINK Bioink. Since bioprinting is a highly customizable technique, parameters described in this method (i.e., cell-cell ratio, scaffold dimensions) can easily be altered to serve other applications, including studies on production of 3D bioprinted THC free Cannabis seeds.

The bio-ink also contains extracellular matrix of the THC-free strain of Cannabis. As the genetically modified stem cells would grow into a callus, via a callus culture. A callus is an unspecialized, unorganized, growing and dividing mass of cells. It is produced when explants (here we refer to genetically engineered THC free plant cells) are cultured on the appropriate solid medium, with both an auxin and a cytokinin in correct conditions. The artificial seeds (embryos) derived from genetically engineered explants will form the compositions to make the bio-ink which is then needed for the 3D printing of proprietary shaped seeds.

This callus tissue could then be used to induce somatic embryogenesis (see FIGS. 4 and 5). Somatic embryogenesis is a developmental process where a plant somatic cell can dedifferentiate to a totipotent embryonic stem cell that has the ability to give rise to an embryo under appropriate conditions. This new embryo can further develop into a whole plant. Not all new embryos may develop into a plant so we would need to validate this first before we could use the contents or compositions including its vascular networks of this validated embryo to be used as the bio-ink for making the proprietary 3D printed seeds with unique shapes. These printed seeds with unique shapes would be proprietary as they are neither obvious nor naturally occurring. Printing the seeds with 3D scaffolds to allow them to develop into proper stem and root vascular structures could be proprietary too.

The generated GM THC Free marijuana plant stem cells and other cellular biomaterials from the embryo generated from the callous tissue grown from successfully genetically engineered high producing cannabinoid strains of marijuana plant stem cells can be used as BioiInks and Biomaterials (the “softwares”) to create the 3D bioprinted THC Free Strains of Marijuana seed/pod.

THC Free strains of Marijuana using 3D Bioprinting creating distinct shapes of seeds over traditional seeds, as a distinct mark easily identifiable to regulatory bodies that proves that indeed these are THC free Cannabis.

Seeds can be printed in any shape, size or color e.g. square instead of oval, or pink instead of normal seed colour.

Seeds includes any plants stem cells or cellular materials that can regenerate and grow into a new plant.

Printing the scaffold, germination arrays and seeding system as one seed microenvironment system (the ‘hardwares’) with bioinks containing soil compositions (as above) and precursor cells (apical meristems, lateral meristems and vascular system) and plant growth regulators in a proportion of 80% auxins and 20% cytokinnins

FIGS. 7, 8 and 9 show the 3D bio-printing process. The following layers be achieved, i.e. 3 layers of scaffolds and arrays (the “hardwares”):

Layer 1: 70% plant growth regulators, 20% precursor cells (80% apical meristems, 20% vascular system cells) and 10% soil compositions.

Layer 2: 20% plant growth regulators, 30% precursor cells (50% lateral meristems, 50% vascular system cells) and 50% soil compositions.

Layer 3: 10% plant growth regulators, 20% precursor cells (80% apical meristems, 20% vascular system cells) and 70% soil compositions.

Bio-Ink (the “softwares”) (to print 5 layers):

Innermost layer: Embryoid cell mixture containing the plant growth including genetically modified DNA instructions (70%) plus apical meristem cell mixture (10%) plus lateral meristem mixture (10%) plus vascular system (including cambium) cell mixture (10%)

Layer next to embryo: Carpel cell mixture

Layer next to carpel: Cupule cell mixture

Layer next to cupule: Calyx cell mixture

Layer next to calyx: Stipule cell mixture

This bioprinter is displaying the temperature (5 degree Celsius to 25 degree Celsius), pressure (1 to 120 PSI) and drops/nozzle (1-10,000 droplets per second) settings just above the three buttons. Resolution/droplet size, we have used 10 micrometers to 1 millimeters.

The following describes the various parts of the bioprinter:

Print head mount—On a bioprinter, the print heads are attached to a metal plate running along a horizontal track. The x-axis motor propels the metal plate (and the print heads) from side to side, allowing material to be deposited in either horizontal direction.

Elevator—A metal track running vertically at the back of the machine, the elevator, driven by the z-axis motor, moves the print heads up and down. This makes it possible to stack successive layers of material, one on top of the next.

Platform—A shelf at the bottom of the machine provides a platform for the organ to rest on during the production process. The platform may support a scaffold, a petri dish or a well plate, which could contain up to 24 small depressions to hold organ tissue samples for testing. A third motor moves the platform front to back along the y-axis.

Reservoirs—The reservoirs attach to the print heads and hold the biomaterial to be deposited during the printing process. These are equivalent to the cartridges in your inkjet printer.

Print heads/syringes—A pump forces material from the reservoirs down through a small nozzle or syringe, which is positioned just above the platform. As the material is extruded, it forms a layer on the platform.

Triangulation sensor—A small sensor tracks the tip of each print head as it moves along the x-, y- and z-axes. Software communicates with the machine so the precise location of the print heads is known throughout the process.

Microgel—Unlike the ink you load into your printer at home, bioink is alive, so it needs food, water and oxygen to survive. This nurturing environment is provided by a microgel—think gelatin enriched with vitamins, proteins and other life-sustaining compounds. Researchers either mix cells with the gel before printing or extrude the cells from one print head, microgel from the other. Either way, the gel helps the cells stay suspended and prevents them from settling and clumping.

Bio-Inks Used: Two Proprietary Seed Related Bio-Inks as described above. ‘Hardware’ bio-ink for printing seed microenvironment integrated system with scaffolds and germination arrays etc. ‘Software’ bio-ink for printing the actual seeds with growth capabilities to germinate into plants, containing embryoid cell materials and other meristem and vascular stem cell materials. Using the 2-bio-inks (‘software’ and ‘hardware’ types) above, we could always program the 3D bio-printer to print seeds according to the proprietary shapes or colours that are desired, these specific features that are completely different from the wild type ones.

In an example a 3D bioprinter is shown in FIG. 6. It has a print head for printing the cellular bio-ink 5 and hydrogel 10, a heating 15 and cooling 20 station, a reservoir for containing the bio-ink, a glass capillary 30, a laser calibration module 35, and a print stage 40. An emergency stop button 45 is also included.

### CONCLUSION

The uniqueness is that our 3D printed seeds contains not just the genetically modified cell contents but also the embryoid materials needed for the seed to germinate into full grown plants. In particular, we have produced permanent transformation of THCA synthase expression in the our unique seeds and we have carried out verification assays to demonstrate that.

Traditional 3D printing only print scaffolds like the one we have given in the green portion, which is not inventive in itself but it is necessary for our printed seeds to have a seed microenvironment built as molecular scaffolds to support its growth subsequently as a germinating seed.

The benefits of creating artificial seeds include the following:

Easy handling—during storage, transportation and planting, as these are of small size.

Inexpensive transport—reason behind is small size.

Storage life—much longer, seed viability remains good for longer time period.

Product uniformity—as somatic embryos used are genetically identical.

To avoid extinction of endangered species—e. g. in hedgehog cacti (Echinocereus sp.)

Large scale propagation—very much suitable for large scale monoculture.

Mixed genotype plantations—suitable for this too, as for monoculture.

Germplasm conservation—important in germplasm conservation.

Elite plant genotypes—artificial seed technology preserves/protects and permits economical mass propagation of elite plant genotypes.

Not a season dependent technology

Permits direct field use—rooting, hardening is necessary as it is in tissue culture plants. It permits direct field sowing.

Facilitates study of seed coat formation, function of endosperm in embryo development and seed germination, somaclonal variation.

Supply of beneficial adjuvants—beneficial adjuvants like plant nutrients, plant growth regulators, microorganisms, fungicides, mycorrhizae, antibiotics can be made available to the developing plant embryo as per the requirement as these can be added in to the matrix.

Propagation of plants unable to produce viable seeds.

Hybrid production—synthetic seed production technology can be used for production of hybrids which have unstable genotypes or show seed sterility. It can be used in combination with embryo rescue technique. The rescued embryo can be encapsulated with this technique.

Easy Identification and tagging—can introduce tracer/markers eg. visible dye/fluorescent markers/microchip for easy tagging and identification.

(1) True seeds are produced in plant at the end of reproductive phase by the process of complex sexual reproduction. A plant may take a long or short time to attain the reproductive phase. So we have to wait up to the end of reproductive phase of a plant for getting seeds. But artificial seeds are available within at least one month. Nobody has to wait for a longtime.

(2) Plants bear the flower and produce the seeds at particular season of a year. But the production of artificial seed is not time or season dependent. At any time or season, one may get the artificial seeds of a plant.

(3) Occasionally, the work on some plants is delayed due to presence of long dormancy periods of their seeds. By growing artificial seeds, this period may be reduced. Using artificial seeds, the life cycle of a plant could be shortened.

(4) Somatic embryogenesis has been observed in a great many species to date, which indicates that it may be possible to produce artificial seeds in almost any desired crops Successful results have already been obtained in some crops such as Apium graveolens Daucus carota, Zea mays, Lactuca satxva, Medicago sativa, Brassica sp. Gossypium hirsutum.

(5) Artificial seeds will be applicable for large-scale monocultures as well as mixed-genotype plantations.

(6) It gives the protection of meiotically unstable, elite genotypes.

(7) Artificial seed coating also has the potential to hold and deliver beneficial adjuvants such as growth promoting thizobacteria, plant nutrients and growth control agents and pesticides for precise placement.

(8) Artificial seeds help to study the role of endosperm and seed coat formation.

Advantage over genetic engineered mutants. Same shapes, very hard to differentiate THC free strains from non THC free strains as shape would be the same.

Even the best authentication methods to identify such strains is only preventive and deterrent in nature but not an absolute assured solution.

Whilst there has been described in the foregoing description preferred embodiments of the present invention, it will be understood by those skilled in the technology concerned that many variations or modifications in details of design or construction may be made without departing from the present invention.

1. A process for producing a genetically modified Cannabis seed that germinates into a plant, the process comprising:
(a) preparing a cell culture comprising genetically engineered Cannabis cells having at least one gene that expresses a psychoactive cannabinoid deleted;
(b) establishing a callus culture for forming a somatic embryo;
(c) forming a bio-ink comprising the somatic embryo; and
(d) three-dimensional (3D) printing the seed.

2. The process according to claim 1, wherein step (a) comprises obtaining cells from a wild-type Cannabis plant and genetically deleting the at least one gene that express psychoactive cannabinoids, wherein the Cannabis plant contains a high level of CBDV content.

3. The process according to claim 2, wherein the at least one gene that expresses a psychoactive cannabinoid deleted is a gene that encodes for a psychoactive cannabinoid selected from the group consisting of THCA, THC, THCVA and THCV.

4. The process according to claim 3, wherein the gene is the THCA synthase gene.

5. The process according to any one of the preceding claims, further comprising the step of replacing the at least one gene that expresses a psychoactive cannabinoid with a reporter gene.

6. The process according to claim 5, wherein the at least one reporter gene further comprises a detectable label.

7. The process according to any one of claim 5 or 6, wherein the reporter gene is the firefly luciferase gene.

8. The process according to any one of the preceding claims, further comprising encapsulating the somatic embryo prior to the 3D printing step (d).

9. The process according to any one of the preceding claims, wherein the step (d) prints a seed having a shape other than that of a naturally occurring wild type Cannabis seed.

10. A genetically modified Cannabis seed that germinates into a plant, wherein the seed has a shape other than that of a naturally occurring wild type Cannabis seed.

11. A plant produced from a seed produced by a process according to any one of claims 1 to 9, or from a seed according to claim 10.
